(19) United States
(12) Patent Application Publication
(10) Pub. No.: US 2006/0260001 A1
(43) Pub. Date: Nov. 16, 2006

(54) Method for obtaining recombinant plants of the cichorium genus and plants thus obtained

(76) Inventor: Alain Lecompte, Angers (FR)

Correspondence Address: YOUNG & THOMPSON, 745 SOUTH 23RD STREET, 2ND FLOOR, ARLINGTON, VA, 22202, US

(21) Appl. No.: 10/554,282

(22) PCT Filed: Apr. 23, 2004
(86) PCT No.: PCT/FR04/50172

§ 371 Date: Oct. 25, 2005

(30) Foreign Application Priority Data
Apr. 25, 2003 (FR) 0305126

(51) Int. Cl. A01H 1/00; C12N 15/82

(52) U.S. Cl. 800/278; 800/279

(57) ABSTRACT

The present invention relates to a method for obtaining recombinant plants of the Cichorium type, adapted to forcing culture and having a phenotype determined through the expression of a combination of phenotype characters derived from Cichdrium intybus L and of Cichorium endivia L, respectively.

The present invention relates to a method for obtaining recombinant plants of the Cichorium genus, adapted to forcing culture and having a phenotype determined through the expression of a combination of phenotype characters derived from Cichorium intybus L and of Cichorium endivia L, respectively.

Plants from Cichorium intybus L species adapted to forcing culture, also called endives, are widely grown throughout Europe and temperate areas in Asia. It is a very branchy plant, with stiff branches being spaced apart from one another. The lower leaves are usually divided, with lobes or segments arranged on both sides, spaced apart and often inverted, with an end lobe; the top leaves are entire, encompassing the stem with their base, and the higher leaves are reduced to relatively small bracts. The root parenchymal cells produce holosides (starch, inulin, etc) from substances originating from green organs where they have been synthetized. During the growth phase, roots constantly bulge out as a result of an hyperplasia of their parenchymae, with holosides accumulating therein, so as to form tuberous roots.

The ability of the Cichorium intybus L species, and more specifically of some varieties of such a species, such as the Witloof type varieties, to produce tuberous roots makes such plants adapted to being grown in the dark through <<forcing>>. The cultivation, for a 21 day forcing, comprises growing endives in tubs containing a nutriment solution brought to a temperature around 18° C.-21° C. with, simultaneously, an air temperature being 1° C. to 3° C. lower than the nutriment solution temperature. Endives are grown through forcing in the dark so as to lead the leaves to wilt in order to produce essentially white mature plants with the outer edge of the leaves only having a slight yellow colour.

Endive producers are seeking novel products for diversifying their offer and allowing them to offer a varied range of endives to the final consumers.

Producing endives through forcing traditionally occurs at a large scale in rooms where tubs are stacked wherein endive roots are placed being immerged in a nutriment solution, under dark conditions, under solution and room temperature conditions, and under accurately controlled hygrometry conditions. Thus, forcing cultivation of endive requires significant financial investments both in equipment and labour in order to achieve the various respective steps of sowing, root harvesting, and their forcing in a cultivation room, in order to produce endives at a large scale.

Cultivating endives through forcing is intended to produce chicories that will be marketed. Chicories are essentially made of leaves wilting in the dark, originating from the root neck. Forcing cycles are subjected to highly accurate and planned cultivation conditions, cycles and schedules so as to optimize the production costs.

In order to make available for the consumers a novel and varied range of plants derived from endive, able to be marketed at reasonable costs, it is required that the novel plants should be adapted to the production conditions being traditionally used for the endive. Indeed, new industrial investments that should be specifically adapted to the production of new plants of the Cichorium species would not be economically compatible with the market. Any new variety intended to be produced through forcing should, consequently, be able to integrate in the production system already developed for the endive.

As far as the Applicant knows, it has never been described in the prior art any recombination method between Cichorium Intybus L with tuberous roots and Cichorium endivia L leading to the production of commercially useful plants and having a combination of phenotype characters expressed from genetic characters of each of the initial parent lines.

Inter-specific hybrids of F1 generation between Cichorium intybus L and Cichorium endivia L have already been described in the prior art. One can more particularly note the hybridization implemented in 1953 by Charles RICK (1953, Proceedings of the American Society for Horticultural Science, vol. 61: 459-466), who has described hybridization events on a field between both these two species. RICK also describes obtaining F2 generation plants produced through self-fertilization of F1 generation plants on a field. RICK observes a very large variability of phenotype characters expressed by F2 generation plants, notably regarding their sturdiness. The above-mentioned author concludes from this that numerous combinations of genes originating from chicory and from endive respectively were not “harmonious” as they considerably reduce the fertility and sturdiness level. According to RICK, there is a nearly impassable genetic barrier between Cichorium intybus L. and Cichorium endivia, although some exchange of genes between both species could be exceptionally observed (see page 464 in Rick, 1953). RICK also observes that the differences between both genomes, of Cichorium intybus L and of Cichorium endivia L respectively, are such that they generate imperfect meioses and sterilities in the descendants through self-fertilization.

F1 generation hybrid plants of the “Castel Franco” type have long been considered as the sole example of gene exchange between both those species, through accidental hybridization, in the 16th century in Italy (Annemieke M Kiers: Endive, Chicory and their wild relatives, Gorteria Supplement 5, Jul. 2000). Recent molecular studies (Kiers, 2000) show that both species are not related and that the Castel Franco type is related to Cichorium intybus L.

It has also been described in the prior art a F1 generation hybrid between Cichorium intybus L and Cichorium endivia L, that has been subsequently propagated exclusively in vitro through cloning, in particularly for embryogenesis study purpose (Blervaq A. S. et al., 1995, Protoplasma, vol. 186:163-168). It has also been disclosed that F1 generation hybrid plants between Cichorium intybus L and Cichorium endivia L have a phenotype character of resistance to the turnip mosaic virus (TuMV), probably through the dominance of the TuMV resistance gene originating from Cichorium intybus L (Providenti et al., 1979, J. Amer. Soc. Hort. Sci., vol. 104 (6): 726-728).

As can been seen from the foregoing analysis, to the knowledge of the Applicant, no chicory to be cultured by forcing, which would be derived from the genetic recombination between Cichorium intybus L and Cichorium endivia L has ever been disclosed to date. There was even a technical prejudice from those skilled in the art against successfully obtaining recombinant plants between both those species, and more particularly recombinant plants susceptible to consist of intermediary products for the purpose of selecting recombinant varieties with tuberous roots, adapted to forcing cultivation and having sturdiness and fertility technical features allowing them to be industrially grown as vegetable plants.

To date, it has never been disclosed any method for obtaining recombinant plants of the Cichorium type originating from an initial cross-breeding between both these those species, of which the order and type of the method steps would have enabled to reach such an objective.

Such a method for obtaining recombinant plants between Cichorium intybus L and Cichorium endivia L with tuberous roots, and adapted to forcing cultivation, is from how on, provided according the invention.

More specifically, the Applicant has tried to develop a method for obtaining such recombinant plants of the Cichorium species, comprising both cross-breeding and field cultivation steps as well as in vitro cultivation and in vitro cloning steps. In particular, the cultivation and in vitro cloning steps, being essential to achieving the method of the invention, allow the overcome the numerous technical problems encountered due to a massive infection of first generation recombinant plants through various bacterial and fungal pathogens.

An object of the invention is therefore a method for obtaining a recombinant plant of the Cichorium type, having tuberous roots, characterized in that it involves the steps of:

a) performing a cross-breeding between a batch of female plants of a variety of the Cichorium intybus L species having tuberous roots and a batch of male plants of a variety of the Cichorium endivia L species and obtaining an F1 generation hybrid plant population resulting from said cross-breeding;

b) performing a self-fertilization of F1 generation hybrid plants resulting from step a) and obtaining F2 generation recombinant plants derived from said cross-breeding;

c) selection of F2 generation recombinant plants which buds or roots do not have any visible alterations caused by a viral, bacterial or fungal infection, particularly by Erwinia carotovora, by Sclerotinia Sclerotiorum, or even by Phytophtora cryptogea;

d) forcing F2 generation recombinant plants selected in step c) for a period of 10 to 18 days under the following forcing conditions:

nutriment solution temperature: 15° C. to 17° C.;

room temperature: 15 to 17° C.;

e) cloning F2 plants obtained at the end of step d) and obtaining regenerated buds;

f) pricking out regenerated buds on an appropriate culture medium until recombinant young plants are obtained.

In step a) of the method, a “manual” cross-breeding is performed between a batch of female plants of a variety of the Cichorium intybus L species having tuberous roots and adapted to forcing and a batch of male plants of a variety of the Cichorium endivia L species.

Any variety of the Cichorium intybus L species could be used in step a) of the method.

In particular, those skilled in the art could advantageously make use of the Cichorium intybus L variety called <<VIDENA>>, being adapted to hydroponic forcing. The VIDENA variety is available to the public from the Collection Nationale du Groupe d'Etudes et de contrôle des Variétés Et des Semences (GEVES, Domaine de la Boisselière, 49250, Brion, France), under the reference <<Ref 500>> and the access number no. 925.

In step a) of the method, any variety of Cichorium endivia L may be used. However, those skilled in the art could advantageously use the <<Grosse Pommant Seule>> line, being available to the public from the Collection Nationale du GEVES (Brion, France) under the reference <<Ref 13746>> and the access number no. 693.

At the end of step a), a batch of F1 generation hybrid plants is obtained resulting from the hereinabove described inter-specific cross-breeding.

In step b) of the method, a cross-breeding is achieved, through self-fertilization of F1 generation hybrid plants obtained in step a).

Self-fertilization advantageously occurs conventionally, in a closed enclosure and in the presence of insects, for example, flies, the self-fertilization then being carried out through said insects conveying male gametes taken from flowers of F1 generation hybrid plants up to the female reproductive organs of neighbouring F1 generation hybrid plants, with which male gametes are contacted. Those skilled in the art could advantageously make use of the conventional self-fertilization technique as described by Hayes et al. (Methods of plant breeding, Mc Grow-Hill Book Company, 1955, Chapter 5, pp 80-93) for the onion, such a technique being directly applicable to the self-fertilization of plants of the Cichorium species.

F2 seeds are obtained resulting from the self-fertilization of F1 generation hybrid plants.

At the end of step b) of the method, F2 generation recombinant plants are obtained, from the above-mentioned F2 seeds.

At the end of step b), the Applicant observed that, very quickly, numerous F2 generation recombinant plants were deficient and died. Numerous other F2 generation plants were found to be very sensitive to various bacterial or fungal pathogens, particularly to Erwinia carotovora bacteria and to Sclérotinia sclerotiorum and Phytophthora cryptogea fungi.

Indeed, lifting roots of F2 generation plants obtained at the end of step b) of the method showed that the roots of Cichorium endivia L as well as the material originating from the inter-specific cross-breeding was more sensitive to Sclerotinia sclerotiorum than the roots of Cichorium intybus L. The roots of Cichorium intybus L were in turn contaminated by the neighbouring diseased plants.

Consequently, step c) of the method consisted of selecting F2 generation recombinant plants obtained at the end of step b) for which buds or roots do not show any alteration visible to the naked eye caused by a viral, bacterial or fungal infection, specifically, by Erwinia carotovora, by Sclérotinia sclerotiorum or even by Phytophthora cryptogea.

In step d), the roots of F2 generation recombinant plants selected in step c) were cultivated through forcing under non classical conditions being specifically adapted so as to obtain, at the end of step d) of forcing, F2 generation small recombinant plants substantially free of any bacterial or fungal infection.

The specific conditions in step d) of forcing consist in the combination of technical characteristics of duration and of temperature.

Under the prescribed temperature conditions, the Applicant observed that step d) of forcing could be performed for a period ranging from 10 to 18 days of forcing, such a period allowing both to obtain sufficiently developed young plants to be cloned in next step e) while remaining substantially free of any visible alterations to the naked eye caused by infections by pathogens, more particularly bacteria or fungi. The best results are achieved when the duration of step d) of forcing ranges from 11 to 17 days, even more advantageous results being obtained with a forcing period ranging from 12 to 16 days. For achieving optimum growth conditions of recombinant young plants without any significant alteration due to a bacterial or fungal infection, the duration of the forcing step preferably ranges from 13 to 15 days and most preferably is 14 days, instead of a conventional 21 day forcing cycle.

The forcing step d) is achieved with a conventional forcing nutriment solution being maintained at a temperature ranging from 15° C. to 17° C., preferably ranging from 15.5° C. to 16.5° C., the optimum temperature being 16° C., instead of temperatures ranging from 18° C. to 21° C. applied for a conventional forcing culture. Simultaneously, the room temperature, i.e. the ambient temperature, is the same as that to which the nutriment solution is maintained, i.e. ranging from 15° C. to 17° C., advantageously from 15.5° C. to 16.5° C. and most preferably being 16° C.

The short duration and the particularly low temperatures used for the forcing step d) allowed, surprisingly, to obtain young plants with characteristics of satisfactory sanitary quality so that such young plants are subsequently in vitro cloned.

The other conditions of the forcing step d), in particular, the nutriment solution composition, are classical. These are described, in particular, by Leteinturier et al. (L'endive-Guide Pratique. édition du CTIFL, September 1991, pages 71.

Preferably, according to the method of the invention, after selection of the F2 generation hybrid plants in step c), the forcing step d) is preceded by a step c1) wherein roots selected in step c) are treated by immersion in a disinfecting solution containing a combination of anti-bacterial and anti-fungal agents. Advantageously, those skilled in the art make use of procymidon (liquid Sumisclex, marketed by the SOPRA company), particularly at a final concentration of 60 g/hL, for inhibiting the growth of Sclerotinia, and to mancozebe (Dithane D G, marketed by the ROHM & HAAS company), in particular at a concentration of 400 g/hl, for inhibiting the growth of Phytophtora.

Following the hereinabove forcing step d), the thus obtained young chicories are advantageously selected before the next cloning step e), based on criteria of expression of phenotype characters encountered in the very varied and little homogeneous population of F2 generation recombinant plants.

Thus, the F2 generation young plant population obtained at the end of the forcing step d) is very heterogeneous. Such a F2 recombinant population comprises young plants with very varied phenotype characters, some young plants having phenotype characters such that they are more related to the parent line Cichorium intybus L, others having phenotype characters such that they are more related to the parent line Cichorium endivia L.

However, all the F2 generation recombinant young plants being subjected to the forcing step d) after selection share in common possessing tuberous roots, making them adapted to a forcing cultivation which is the object to be achieved by the invention. The recombinant character of possession of tuberous roots was initially provided by the genome of the parent Cichorium intybus L. All the selected F2 generation recombinant young plants also have in common possessing indented leaves. The recombinant character of possession of indented leaves was initially provided by the genome of the parent Cichorium endivia L. Such a combination of phenotype characters that is possessed by all the selected F2 generation recombinant plants which were obtained according to the method of the invention, allows to define, generally speaking, the recombinant plants obtained at the end of step f) of the method, as well as the recombinant plants able to be obtained following additional steps of the method, such as steps g) to j) which are described later in the present specification.

Generation F2 plants obtained as early as the end of step b) of the method according to the invention are recombinant plants, i.e. plants, which, after multiple recombination events between the DNA originating from Cichorium intybus L. and the DNA originating from Cichorium endivia L., possess a recombined genome of the same ploidy as the ploidy of each of both parent plants. The recombined genome of F2 generation plants according to the invention thus expresses combinations of phenotype characters, some phenotype characters being initially encountered in Cichorium intybus L., and some phenotype characters being initially encountered in Cichorium endivia L.

Thus, according to the method, the forcing step d) is followed by a step d1) wherein the resulting young chicories are selected before the cloning step e), according to three following phenotype classes:

(i) PPI: very numerous narrow leaves on a plate-shaped root neck;

(ii) GPI: typology similar to the endive, but with a narrow and indented leaf; and

(iii) TFR and SCA: very dentate branched leaves.

All F2 generation recombinant plants according to the above-described phenotype class (iii) have axes secondary to the main axis of the leaf, as this is illustrated in the general diagram on FIG. 1, which is a characteristic never observed with Cichorium intybus L. In some endive genotypes, such as endives of the “Barbe de Capucin” (wild chicory) genotype, it can be observed that the edge of the limb could show shallow indentations, i.e. of which the ratio between (i) the indentation depth and (ii) the length between the indentation tip and the leaf axis that does not exceed 0.25 (see FIG. 1) while such a ratio is considerably much higher in F2 generation recombinant plants according to the invention. Moreover, in endive genotypes such as the Barbe de Capucin, the indentations are only present in the upper third of the leaf.

The three classes of F2 generation recombinant plants obtained in a reproducible way at the end of step d) of the method according to the invention are further described herein below, referring to the measurement of several phenotype characters allowing to define them, being represented on FIG. 1, and being respectively the following characters:

Main axis (1) and secondary axes of the leaf (2);

Width of the leaf basis (3);

Height of the leaf (4);

Depth of the indentation (5) and length between the indentation tip to the leaf axis (6);

Presence of a secondary serration (7).

Description of the PPI Recombining Plant Class:

more than 100 leaves per root at the completion of the forcing (recombining character originating from C endivia L. ),versus 20 to 35 maximum for a traditional endive;

no secondary axis;

very narrow basis of each leaf:

ratio width of the leaf basis/height of the leaf ranging from 0.06 to 0.10 compared to 0.25 minimum for a traditional endive;

deep indentations of the limb:

ratio depth of the indentation/length of the indentation tip to the leaf axis ranging from 0.60 to 0.85, compared to a ratio ranging from 0 to 0.25 for some genotypes of C intybus L.;

The edge of the indentations comprises or not secondary serrations;

the colour of the nervures is white or red;

the colour of the limb is yellow or red;

Description of the GPI Recombining Plant Class:

from 20 to 35 leaves obtained per root at the completion of the forcing, as for a traditional endive;

No secondary axis;

Deep indentations of the limb up to the leaf basis:

ratio depth of the indentation/length of the indentation tip to the leaf axis ranging from 0.60 to 0.85, compared to a ratio ranging from 0 to 0.25 for some genotypes of C intybus L.;

The edge of the indentations comprises or not secondary serrations;

the colour of the nervures is white or red;

the colour of the limb is yellow or red;

Description of the TFR and SCA Recombining Plant Class:

Description of the TFR Product Class:

from 20 to 35 leaves obtained per root at the completion of the forcing, as for a traditional endive;

2 to 5 axes secondary to the main axis starting in the basal half of the leaf;

Deep indentations of the limb: ratio depth of the indentation/length of the indentation tip to the leaf axis ranging from 0.60 to 0.85;

The edge of the indentations comprises or not secondary serrations;

The colour of the nervures is white or red;

The colour of the limb is yellow or red;

Description of the SCA Product Class:

The definition is similar to that of recombining plants of the TFR class.

The indentations are shallow and the serrations nearly nonexistant.

As for the TFR, the major characteristic is the presence of secondary axes (characteristics only encountered for some C endivia L. and never for C intybus L.)

The population of F2 recombinant plants, as well as the sub-populations of F2 recombinant plants selected according to the three hereinabove described phenotype classes (i) to (iii) are not homogeneous in the expression of phenotype characters other than those mentioned hereinabove. For example, the F2 recombining plant sub-populations (i) to (iii) comprise in turn a wide phenotype variety between themselves, it being understood that they however possess in common the phenotype characters on the basis of which they were classified in the same class.

Moreover, the Applicant could observe that the expression of phenotype characters, other than those on the basis of which the different F2 plants were selected, was not stable throughout the successive generations, i.e. throughout successive reproduction cycles, including through self-fertilization of the above defined sub-populations (i) to (iii).

In step e) of the method, the F2 generation recombinant plant population or even the sub-populations of hereinabove defined phenotype classes (i) to (iii), are cloned so as to obtain regenerated buds.

The phenotype selection and cloning of F2 recombinant plants after forcing and, optionally phenotype selection, occur conventionally, for example, on a culture medium adapted for regenerating buds, such as the “ER” medium from fragments of leaf nervures taken from young chicories obtained at the end of the forcing step d), optionally on the F2 plants previously selected in phenotype classes (i) to (iii), for example, according to the technique described by Margara (Margara J, 1989, Bases de la multiplication végétative, INRA ed.). Preferably, the fragments of leaf nervures are desinfected with calcium hypochlorite before being cultivated with a view to regenerating buds on the appropriate culture medium.

The cloning step of the F2 recombinant plants, optionally after they were selected according to the above mentioned phenotype classes (i) to (iii), allows for the prophylactic control of a possible infection of the young plants resulting from the forcing by bacterial or fungal pathogens. Indeed, the Applicant could observe that the young chicories obtained at the end of the forcing step d) were substantially sensitive to an infection through Sclerotinia sclerotinorum, even when the forcing step was preceded by a treatment of the roots using a nutriment solution containing a combination of antibacterial or antifungal agents, as described herein above.

The cloning step e), more particularly when it comprises a disinfection of the fragments of leaf nervures prior to the culture on a culture medium with a view to regenerating buds, allows to considerably improve the sanitary condition of the cloned vegetal material.

The cloning step e) is followed by a pricking out step of the thus regenerated buds on an appropriate culture medium, under conventional bud pricking out conditions, until F2 generation hybrid young plants are obtained, cloned and pricked out. The pricking out step f) is conventional by itself and could be performed by those skilled in the art using any technique known as such, for example, on the <<M4>> specific medium (Murashige T and Skoog F, 1962, Physiol. Plant., Vol. 15: 473-497) enriched with AIA at the final concentration of 0.2 mg/l.

For example, those skilled in the art could advantageously make use of the pricking out technique as described in the examples of the present description.

The cloned and pricked out F2 recombining young plants, as obtained at the end of step f), could then be acclimatized, as described in the examples, then rooted and placed in a greenhouse or on a field.

The cloned and pricked out F2 recombining plants making up the final product in step f) of the method of the invention, including the sub-populations of cloned and pricked out F2 recombining plants first selected according to their phenotype characters in the phenotype classes (i) to (iii), respectively form a very heterogeneous population, or three very heterogeneous sub-populations, regarding the expression of their phenotype characters. For example, according to a concrete illustration of the implementation of the method according to the invention, specified in the examples, the cloned and pricked out F2 recombining plants, final products of step f) of the method are made up as follows:

ten F2 recombinant plants of the (i) PPI sub-class sharing in common very numerous narrow leaves on a plate-shaped root neck, but differing there between through numerous other phenotype characters;

eight F2 recombinant plants of the (ii) GPI phenotype sub-class sharing in common a typology similar to the endive with a narrow and indented leaf but differing there between through numerous other phenotype characters.

four F2 recombinant plants belonging to the (iii) SCA phenotype sub-class sharing in common branched leaves but differing there between through numerous other phenotype characters;

three F2 recombinant plants of the (iii) TFR phenotype sub-class sharing in common very dentate branched leaves but differing there between through numerous other phenotype characters; and moreover, the Applicant found out that, out of the 25 F2 recombinant plants obtained at the end of step f) of the method according to the invention, only 19 of them produced seeds after self-fertilization, which means that six F2 recombinant plants out of the nineteen obtained at the. end of step f) of the method are highly self-incompatible.

The method for obtaining a hybrid plant of the Cichorium species, according to the invention is characterized in that it could further comprise the additional following steps:

g) cultivating in the ground small recombinant plants obtained at the end of step f);

h) self-fertilizing F2 recombinant plants as obtained at the end of step g) and obtaining F3 generation recombinant plants through cultivating in the ground.

The populations of F3 recombinant plants are characterized from the phenotype standpoint by their common ability to tuberize, i.e. to develop roots adapted to forcing cultivation.

Advantageously, the F3 generation recombinant plants obtained in the above described step h) are subjected to a forcing step (i), if possible under forcing conditions identical to those implemented in step d) of the method, i.e. a forcing step i) period of ten to eighteen days with a nutriment solution temperature ranging from 15° C. to 17° C., the nutriment solution and the room temperatures being preferably identical.

Advantageously, the above mentioned forcing step i) is followed by a cloning step j) of the F3 generation recombinant young chicories, said cloning step being indiscriminately performed from fragments of leaf nervures or from end buds of F3 generation young plants resulting from the forcing cultivation.

According to a first advantageous alternative, the cloning step j) comprises a cloning step of fragments of leaf nervures of the young plants obtained at the end of the forcing step (i), being followed by a step of regeneration of F4 generation recombinant young plants.

According to a second advantageous alternative, the cloning step j) comprises a step of cloning end buds of young plants obtained at the end of forcing step (i) and regenerating F4 generation recombinant young plants.

As for steps d) and e) of the method, the above mentioned steps i) and j) allow for a control of the sanitary condition of the vegetal material: the combination of technical characteristics of duration and temperature of the forcing step as well as the possibility to “sterilize” the vegetal material used for cloning, for example, using calcium hypochloryte at the final concentration of 2.5% by weight, have the effect of limiting, even of completely inhibiting, possible infections of said recombinant vegetal material by bacteria or fungi.

Preferably, according to the first mode of achievement of the above mentioned cloning step j), generating F4 generation recombinant young plants is performed cultivating fragments of leaf nervures of young plants on a “NF” specific medium as described in the examples.

Preferably, according to an embodiment of the above mentioned cloning step j), regenerating F4 generation recombinant young plants, in step k) of the method, is performed through cultivating end buds on a M4 specific medium, as described in the examples.

Although maintaining a high phenotype diversity, the F4 generation recombinant plants obtained at the end of the above mentioned step j) have an excellent sanitary quality. More particularly, they are free of an infection by Sclérotinia sclerotiorum, Phytophthora or by Erwinia carotovora.

The different phenotype sub-classes (i) to (iv) defined in the present description are encountered in each of the phenotype sub-classes of F4 generation recombinant plants.

It has been shown according to the invention that F4 generation recombinant plants could be easily cultivated through forcing under forcing conditions identical to those conventionally used for Cichorium intybus L, of the Witloof type. The efficiency of the method according to the invention has therefore been shown for obtaining recombinant plants of the Cichorium type having tuberized roots and adapted for forcing cultivation to obtain a final product vegetal material possessing original vegetal typologies unknown until now, in particular, of the final product vegetal material having the following vegetal typologies:

chicories having deeply indented leaves which do not look like endives, nor curly endives.

as tuberized roots as those of endive allowing thereby to meet the production and forcing protocols conventionally used by the endive producers (Cichorium intybus L).

Moreover, it is contemplated fixing the phenotype characters on F4 generation recombinant plants obtained according to the method of the invention by several successive reproduction cycles of each of the F4 generation plants obtained at the completion of the method, through conventional self-fertilization, this with a view to obtaining lines of recombinant plants able to stably express a combination of characters resulting from a genotype or from a combination of genotypes, said characters being expressed homogeneously, so as to impart to some recombinant plants derived from Fn generation recombinant plants (where n represents an integer at least equal to 6) a stable and homogeneous vegetal variety character.

Another object of the invention comprises recombinant plants obtained using the method as defined hereinabove in the present description, said recombinant plants being characterized in that they result from an initial cross-breeding between a female plant from a variety of the Cichorium intybus L species having tuberized roots and a male plant from a variety of the Cichorium endivia L species, and in that they have one of the four following phenotype classes:

(i) PPI: very numerous narrow leaves on a plate-shaped root neck;

(ii) GPI: typology similar to the endive, but with a narrow and indented leaf, or

(iii) TFR and SCA: very dentate branched leaves.

The recombinant plants according to the invention encompass, more particularly:

1) F2 generation recombinant plants obtained at the end of step f) of the method;

2) F3 generation recombinant plants obtained at the end of step h) of the method;

3) F3 generation recombinant plants obtained at the end of step i);

4) F4 generation recombinant plants obtained at the end of step f) of the method.

The present invention is further illustrated by the following figures and examples.

### FIGURES

FIG. 1 represents a theoretical diagram of a wilted leaf of a recombining plant of the Cichorium species according to the invention. Such a theoretical diagram illustrates the various phenotype characters allowing to define a recombining plant of the invention, or one sub-type thereof.

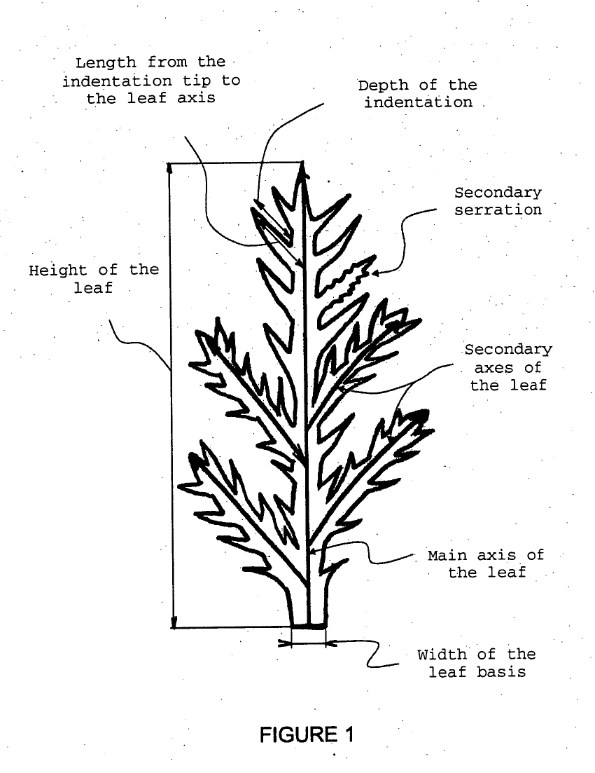

FIG. 2 is a photography of a F4 generation recombining plant having very numerous narrow leaves on a plate-shaped root neck of the PPI phenotype sub-class.

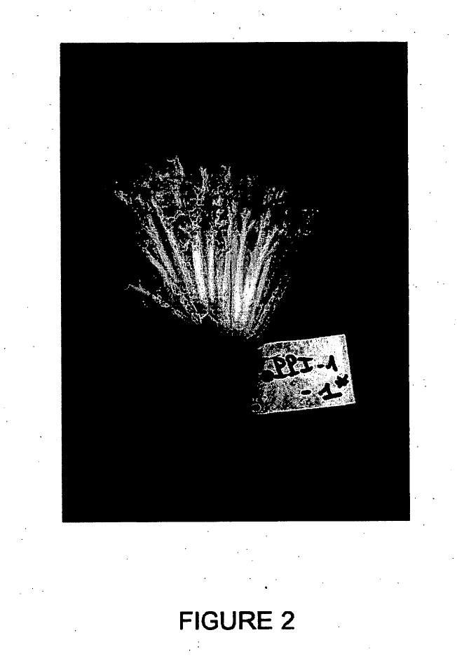

FIG. 3 is a photography of a F4 generation recombining plant with branched leaves of the SCA phenotype sub-class.

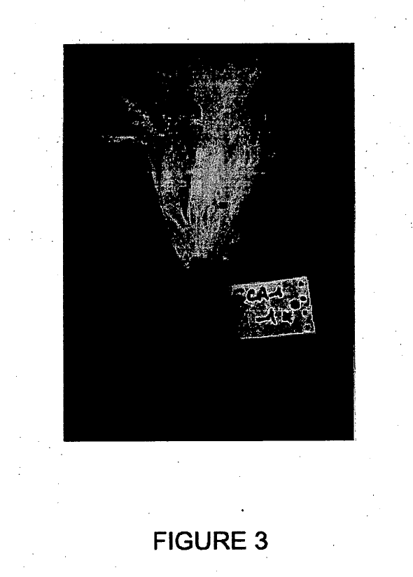

FIG. 4 is a photography of a F4 generation recombining plant with very dentate branched leaves representative of the TFR phenotype sub-class.

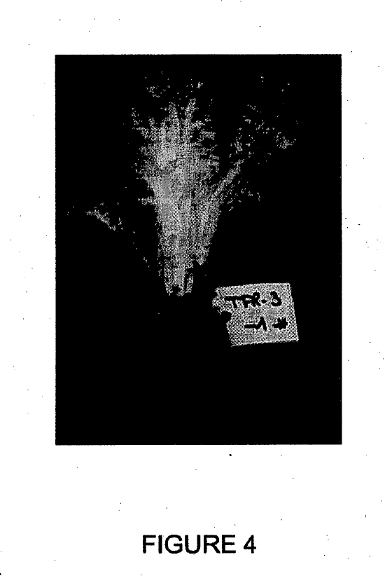

FIG. 5 is a photography of a F4 generation recombining plant with a typology similar to the endive with a narrow and indented leaf representative of the GPI phenotype sub-class.

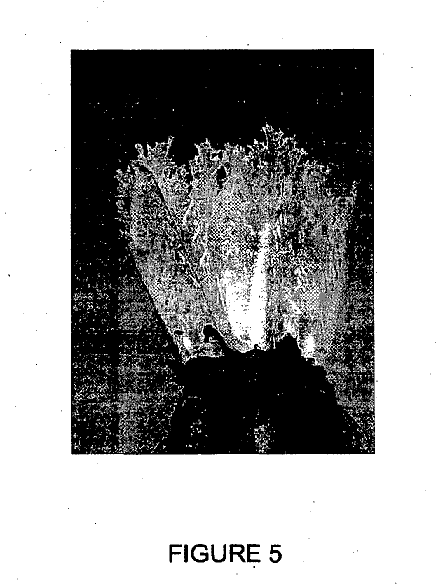

FIG. 6 is a photography illustrating the comparison between four recombining plants of the Cichorium species of the F4 generation GPI type according to the invention (arranged vertically, on the top of the figure) with a plant of the Cichorium intybus L species, also called endive (arranged horizontally, at the bottom of the figure).

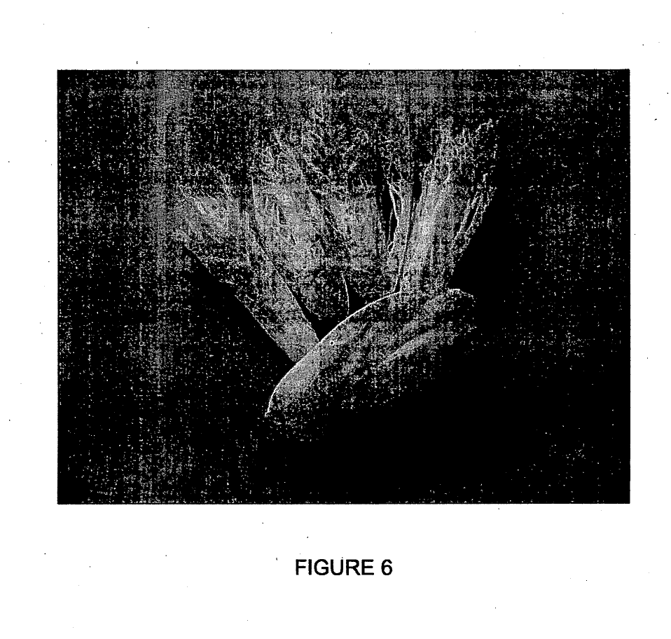

FIG. 7 is a photography showing in detail the indentations of leaves of a recombining plant of the Cichorium species of the F4 generation GPI type according to the invention.

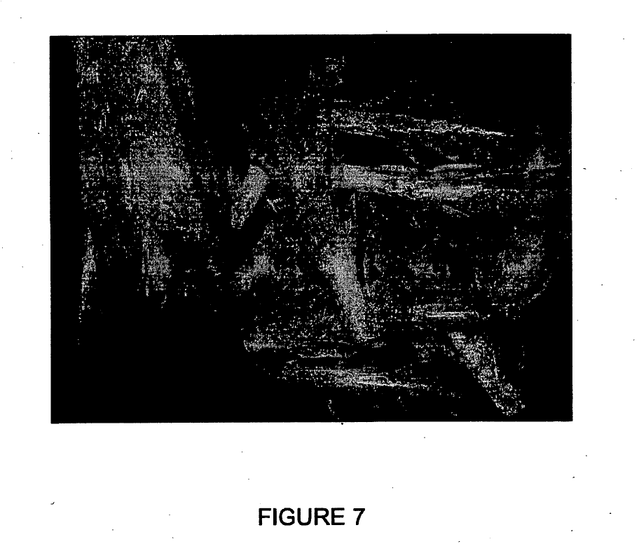

FIG. 8 is a photography illustrating a recombining plant of the Cichorium species of the F4 generation GPI type according to the invention, adapted for forcing cultivation.

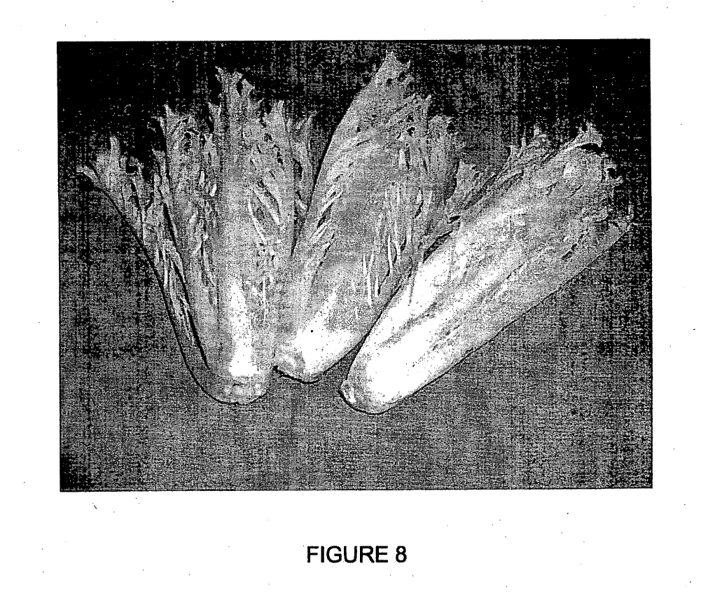

### EXAMPLES

### Example 1

### Obtaining F2 Generation Plants According to Steps a) to f) of the Method of the Invention

A. Material and Methods

A. 1. Culture Media

The qualitative and quantitative composition of the culture media <<NF>> and <<M4>> used in the examples are indicated in tables I and II hereinafter, respectively.

TABLE I
<< NF >> medium
NF (1 liter)

| | | |
|---|---|---|
| MS macroelements (×10) | 100 | ml |
| MS microelements (×1000) | 1 | ml |
| Gamborg vitamins (×1000)* | 1 | ml |
| NaFeEDTA (×100) | 10 | ml |
| Casein hydrolysate | 1 | g |
| Sucrose | 40 | g |
| Inositol | 50 | mg |
| BAP (dissolve in NaOH 1N) | 0.2 | mg |
| pH = 5.6 | | |
| Agar | 10 | g |

*Inositol-free Gamborg vitamins

TABLE II
<< M4 >> medium
M4 (1 liter)

| | | |
|---|---|---|
| MS macroelements (×10) | 100 | ml |
| MS microelements (×1000) | 1 | ml |
| MS vitamins (×1000)* | 1 | ml |
| NaFeEDTA (×100) | 10 | ml |
| Sucrose | 30 | g |
| Inositol | 100 | mg |
| AIA (dissolve in 95° pure alcohol) | 0.2 | mg |
| pH = 5.8 | | |
| Agar | 8 | g |

*Inositol-free MS vitamins

A.2. In vitro Cloning Method of Recombining Young Plants, from Chicory Leaf Nervures.
1) Sampling

Select internal chicory leaves;

Take the leaf nervure:

Place the taken leaves into a desinfecting vessel;

2) Desinfection

The disinfecting step is carried out with a calcium hypochlorite solution at the final concentration of 2.5% +a few drops of Tween® solution for 10 minutes. Then, three successive rincing steps are carried out using sterile water under a laminar flow hood.

3) Culture—Regeneration

Place the nervures on a sterile blotting paper;

Cut out nervure fragments with a surface of about 1 cm

Culture on a NF medium, in an appropriate glass tube;

Place the tubes in a culture room, under the following temperature and luminosity conditions:

Luminosity equivalent to day light for 16 hours, at a temperature of 25° C.;

- Darkness conditions of night time for 16 hours, at a temperature of 20° C.

4) Rooting

After approximately 3 weeks of culture on a NF medium, once the regenerated buds are well developed, the buds are individualized and each young plant is pricked out in tubes on M4 medium, for their rooting.

Place the tubes in a culture room, under the following temperature and luminosity conditions:

Luminosity equivalent to day light for 16 hours, at a temperature of 25° C.;

Darkness conditions of night time for 16 hours, at a temperature of 20° C.

5) Acclimatization

As soon as roots appear, the young plants are acclimatized on compost (in bag), in honeycombed plates with a diameter of 4 cm and at steamed at 20° C.

B. A Method for Obtaining Recombining Plants of the F2 Generation of the Cichorium Species According to the Invention.

a) Manual cross-breeding in a greenhouse of the Cichorium intybus <<1089>> line with the Cichorium endivia <<Grosse pommant seule>> line so as to obtain the F1 hybrid.

Sowing and growing 30 F1 plants up to a 3 leaf stage, followed by a 8 week phase of vernalization at 5° C. so as to obtain the bolting ability.

b) bolting and self-fertilization (in heated greenhouse) in the presence of flies for obtaining F2 seeds.

c) Sowing in the field of 20,000 F2 seeds.

Then lifting roots removing the visibly sick or abnormal plants (advantitious budding, root bursting . . . ).

Treatment of the 10,000 selected roots (60 g/hl of Procymidon and 400 g/hl of Mancozebe).

Storing roots while waiting for forcing in a refrigerator at 0° C.

d) Short 14 day forcing at 16° C.

At the completion of such forcing, nearly all the system was contaminated.

Hence, the impossibility to recover viable plants on their roots..

e) After observations on the leaves parts, 25 plants were selected on the basis of 4 different typologies:

PPI: 10 choices

SCA: 4 choices

TFR: 3 choices

GPI: 8 choices

In order to regenerate such choices, fragments of wilted nervures (1 cm 2) were disinfected using calcium hypochlorite and cultured in vitro on a NF medium. Culture of 24 fragments per choice.

f) Three weeks later, after the infected fragments were removed, the regenerated buds were pricked out on a M4 medium for rooting.

Such a step is followed by a steamed acclimatization step, at 20° C.

### Example 2

### Obtaining F3 Generation Plants According to the Invention, According to Step g) and h) of the Method

The materials and methods are, unless otherwise stated, those being described in Example 1.

g) After 8 weeks vernalization at 5° C., the plant growth occurs in a heated greenhouse.

h) Plants blossomed during the month of April individually under a cloth sleeve, for placing flies therein, and achieving self-fertilizations.

Overall, 19 choices resulted in F3 seeds. The other plants were incompatible.

Treatment and Acclimatization of F3 Generation Recombining Plants Obtained in Step h).

Sowing of F3 descendants is performed on the field.

1) When observing plants growing on the field, the TFR typologies were found to be very deficient (leaf dryness . . . ). A SCA choice showed sclerotinia attacks as early as the field stage.

A brief forcing operation was again to be contemplated, as the root batches carried inoculum.

Lifting roots.

Treatment of the selected roots (60 g/hl of Procymidon and 400 g/hl of Mancozebe).

Storing roots while waiting for forcing in a refrigerator at 0° C.

2) In December, roots were again to be forced within two weeks at 16° C.

3) Very few plants were kept:
PPI: 1 choice
GPI: 2 choices
SCA: 3 choices

In order to regenerate these choices, fragments of wilted nervures (1 cm²) were disinfected using calcium hypochlorite and were cultured in vitro on a NF medium.

Culture of 24 fragments per choice.

4) Three weeks after removal of the infected fragments, the regenerated buds were pricked out on a M4 medium for rooting.

Such a step was followed by an acclimatization step.

### Example 3

### Obtaining F4 Generation Plants of the Invention, According to Steps i) and i) of the Method

The materials and methods are, unless otherwise stated, those being described in Example 1.

a) After 8 weeks vernalization at 5° C., the plant growth occurs in a heated greenhouse.

Plants blossomed during the month of April 1997 individually under a cloth sleeve, for placing flies therein, and achieving self-fertilizations.

b) Then sowing of F4 generations on the field.

At such a F4 stage, the resulting plants did not show deficiencies on the field and the forcing of the harvested roots could be contemplated in an index conventional production scheme (3 weeks at 19° C.). The tricky phase of F2 and F3 generations being over.

c) During forcing, the selected descendants showed a good ability to forcing and phenotype characteristics being the starting point of a diversification of chicories than can be forced.

1. A method for obtaining a recombinant plant of the Cichorium type, having tuberous roots, characterized in that it comprises the steps of:
a) performing a cross-breeding between a batch of female plants of a variety of the Cichorium intybus L species having tuberous roots and a batch of male plants of a variety of the Cichorium endivia L species and obtaining a F1 generation hybrid plant population resulting from said cross-breeding;
b) performing a self-fertilization of F1 generation hybrid plants resulting from step a) and obtaining F2 generation recombinant plants derived from said cross-breeding;
c) selecting F2 generation recombinant plants, wherein the buds or the roots thereof do not have any visible alterations caused by a viral, bacterial or fungal infection, particularly by Erwinia carotovora, by Sclerotinia Sclerotiorum, or even by Phytophtora cryptogea;
d) forcing F2 generation recombinant plants selected in step c) for 10 to 18 days under the following forcing conditions:
nutriment solution temperature: 15° C. to 17° C.;
room temperature: 15 to 17° C.;
e) cloning F2 plants obtained at the end of step d) and obtaining regenerated buds;
f) pricking out regenerated buds on an appropriate culture medium until recombinant young plants are obtained.

2. The method according to claim 1, characterized in that the forcing step d) is followed by a step
d1) wherein the resulting young chicories are selected, before the cloning step e), according to the three following phenotype classes:
(i) PPI: very numerous narrow leaves on a plate-shaped root neck;
(ii) GPI: typology similar to the endive, but with a narrow and indented leaf, and
(iii) TFR and SCA: very dentate branched leaves.

3. The method according to claim 1, characterized in that it comprises the following additional steps:
g) cultivating in the ground small recombinant plants obtained at the end of step f);
h) self-fertilizing F2 recombining plants as obtained in step g) and obtaining F3 generation recombining plants through cultivating in the ground.

4. The method according to claim 3, characterized in that the F3 generation recombinant plants obtained in step h) are subjected to a forcing step i) for a period of 10 to 18 days, under the following forcing conditions:
nutriment solution temperature: 15° C. to 17° C.;
room temperature: 15 to 17° C.;

5. The method according to claim 4, characterized in that it further comprises a cloning step j) of the young recombinant chicories obtained at the end of the forcing step i).

6. The method according to claim 5, characterized in that the cloning step j) comprises cloning the fragments of leaf nervure of young plants obtained at the end of the forcing step i) and regenerating the F4 generation recombinant young plants.

7. The method according to claim 5, characterized in that the cloning step j) comprises cloning the end buds of young plants obtained at the end of the forcing step i) and regenerating the F4 generation recombinant young plants.

8. Recombinant plants obtained by the method according to claim 1, characterized in that they have (i) tuberous roots and (ii) indented leaves.

9. Recombinant plants according to claim 8, characterized in that they belong to class PPI and have the following common phenotype characteristics:
more than 100 leaves per root at the completion of the forcing;
no secondary axis;
very narrow basis of each leaf:
ratio width of the leaf basis/height of the leaf ranging from 0.06 to 0.10;
deep indentations of the limb:
ratio depth of the indentation/length of the indentation tip to the leaf axis ranging from 0.60 to 0.85;
The edge of the indentations comprises or not secondary serrations;
The colour of the nervures is white or red;
The colour of the limb is yellow or red;

10. Recombinant plants according to claim 8, characterized in that they belong to class GPI and have the following common phenotype characteristics:
from 20 to 35 leaves obtained per root at the completion of the forcing;
no secondary axis;
deep indentations of the limb up to the leaf basis:
ratio depth of the indentation/length of the indentation tip to the leaf axis ranging from 0.60 to 0.85;
The edge of the indentations comprises or not secondary serrations;
The colour of the nervures is white or red;
The colour of the limb is yellow or red;

11. Recombinant plants according to claim 8, characterized in that they belong to class TFR or SCA and have the following common phenotype characteristics:
from 20 to 35 leaves obtained per root at the completion of the forcing;
2 to 5 axes secondary to the main axis occurring in the basal half of the leaf;
deep indentations of the limb:
ratio depth of the indentation/length of the indentation tip to the leaf axis ranging from 0.60 to 0.85;
The edge of the indentations comprises or not secondary serrations;
The colour of the nervures is white or red;
The colour of the limb is yellow or red;

12. The method according to claim 2, characterized in that it comprises the following additional steps:
g) cultivating in the ground small recombinant plants obtained at the end of step f);
h) self-fertilizing F2 recombining plants as obtained in step
g) and obtaining F3 generation recombining plants through cultivating in the ground.
